(19) United States
(12) Patent Application Publication
(10) Pub. No.: US 2025/0381250 A1
(43) Pub. Date: Dec. 18, 2025

(54) COLLAGEN MACROPEPTIDE PREPARATIONS AND USES THEREOF

(71) Applicant: BIOVENTUS LLC, Durham, NC (US)

(72) Inventors: Carl Ralph FLANNERY, Raleigh, NC (US); Kelly Elizabeth BUDDIN, Durham, NC (US)

(21) Appl. No.: 19/241,358

(22) Filed: Jun. 17, 2025

Related U.S. Application Data
(60) Provisional application No. 63/661,412, filed on Jun. 18, 2024.

(51) Int. Cl. A61K 38/39; A61K 9/00; A61K 31/12; A61K 31/192; A61K 31/405; A61K 31/407; A61K 31/421; A61K 31/5415; A61K 31/635

(52) U.S. Cl. CPC A61K 38/39; A61K 9/0019; A61K 31/12; A61K 31/192; A61K 31/405; A61K 31/407; A61K 31/421; A61K 31/5415; A61K 31/635

(57) ABSTRACT

The instant disclosure describes an injectable composition comprising a Cathepsin K blocking amount of a polydisperse preparation of collagen macropeptides with an average molecular weight between about 10 and about 100 kDa and an acceptable sterile injection vehicle. Methods of treating arthritis and methods of manufacturing injectable composition are also disclosed.

### FIELD OF THE INVENTION

The present disclosure relates to an injectable composition comprising a polydisperse preparation of collagen macropeptides with an average molecular weight between about 10 and about 100 kDa formulated in an acceptable sterile injection vehicle for blocking of Cathepsin K collagenase. The compositions are useful in treating diseases associated with Cathepsin K collagenase degradation of naturally occurring triple helix collagen in humans and animals.

### BACKGROUND

Osteoarthritis (OA) is the most common type of musculoskeletal disorder and the main cause of aged-related disability. In the United States, the prevalence of OA increases with age and more than 80% of individuals older than 65 years are affected by OA. OA is normally characterized by the loss of articular cartilage, subchondral bone sclerosis and osteophyte formation. This disease has been primarily known as a cartilage disorder associated with focal articular degradation. Cathepsin K (Cat K), a potent collagenase, has been implicated in the breakdown of collagen which contributes to the pathogenesis of OA. See generally, Hayami et al., Inhibition of Cathepsin K reduces cartilage degeneration in the anterior cruciate, ligament transection rabbit and murine models of osteoarthritis, Bone 50 (2012), 125-59. Proof of concept for a beneficial effect of blocking the enzymatic activity of Cathepsin has been demonstrated in clinical studies and in preclinical models of osteoarthritis. Id. See also Bihlet et al., “Symptomatic and structural benefit of cathepsin K inhibition by MIV-711 in a subgroup with unilateral pain: post-hoc analysis of a randomized phase 2a clinical trial” Clin. Exp. Rheumatol., Vol. 40, No. 5, P. 1034-37 (2022). Such studies utilized systemically delivered small molecule inhibitors, for which development by multiple pharmaceutical companies was discontinued due to adverse side-effects in the clinic. See D. Lowe, Cathepsin K: A Promising Target Fades Out, Science Blog 30 Sep. 2016 Cathepsin K: A Promising Target Fades Out|Science|AAAS.

Collagen is the main structural protein in the extracellular space in the various connective tissues in animal bodies. It is found in animals exclusively. Collagen is not a uniform substance but is rather a family of proteins. There are more than 20 types of collagen, varying in structure and occurrence, the most frequent being the fibrillar collagens (typified by collagen types I, II, III, V and XI). Collagen type I is most abundant in the body (in most musculoskeletal tissues such as bones, skin, etc.), although in cartilage, collagen type II is predominant. Fibrillar collagens have a triple helical structure with a molecular weight of approximately 300,000 Daltons (300 kDa) comprised of three distinct polypeptide chains connected via hydrogen bonds. Fibrillar collagens are poorly soluble in cold or hot water. Collagens are characterized by a high content of hydroxyproline and glycine (almost three times the amounts in other proteins), low content of sulfur containing amino acids and absence of tryptophan. This amino acid composition is responsible for the 3-dimensional conformation of collagen. The largest commercial sources of collagen are derived from the connective tissues of cows and pigs (skin, hides, bone), and from fish skins and scales. Collagen is almost always present in by-products of commercial meat processing. Tarantino et al. reported that intra-articular collagen injections may represent a valid therapeutic option for knee OA. See “Intra-articular collagen injections for osteoarthritis: a narrative review” Int. J. Environ. Res. Pub. Health, 2023 March; 20(5): 4390. See also De Luca et al., “Intra-Articular Injection of Hydrolyzed Collagen to Treat Symptoms of Knee Osteoarthritis. A Functional In Vitro Investigation and a Pilot Retrospective Clinical Study”, J. Clin. Med. 2019 July; 8(7); 975.

Gelatin is a mixture of higher molecular weight unwound collagen chains and polydisperse collagen peptides. Gelatin is extracted from collagen sources, usually pretreated with acid or alkali to break down connective structure and to render the material soluble in warm or hot water. Subsequent processing, which may include filtration, ion exchange and drying, yields a dry crystalline granule or powder which is soluble in warmed water but insoluble in cold water. This process also purifies the collagen protein by removing fat, salts, carbohydrates and non-collagen proteins. Thus, the triple helix structure is broken down and replaced by single chains of amino acids (peptide chains) of varying length. Average molecular weights of those peptide chains are typically in the range of s 10,000 Daltons (10 kDa) to approximately 200,000 Daltons (200 kDa). Gelatin can form a gel in aqueous solutions and is used in formulations for pharmaceutical capsules, vaccine stabilization and more generally in the food industry. Gelatin is known to be a substrate for Cathepsin K. Li et al., “Regulation of Collagenase Activities of Human Cathepsins by Glycosaminioglycans”, J. Bio. Chem. Vol. 279, Issue 7, p. 5470-79 (2004).

U.S. Pat. No. 11,040,081 assigned to Genecol Canada, a nutritional supplement manufacturer, entitled “Use of low molecular weight collagen hydrolysate for preventing and/or reducing joint pain, lateral meniscal protrusion and/or improving cartilage abrasion grade” describes the use of collagen hydrolysate with a molecular weight of less than 1500 Daltons (1.5 kDa) in preventing, and/or reducing joint pain in a patient. Collagen hydrolysate is made by enzymatic digestion to hydrolyse peptide bonds of the collagen chains. Selection of enzymes, time, temperature and pH can control digestion of the collagen chains to a mixture of lower molecular weight chains. Collagen hydrolysate contains peptides with different chain lengths or molecular weights, which are produced during the enzymatic hydrolysis. Typical collagen hydrolysates are water soluble and have average molecular weights in the range of 2000-5000 Daltons (2-5 kDa). The term Ultra Low Molecular Weight Hydrolysate may be used for materials below 2000 Daltons (2 kDa). If the low molecular collagen hydrolysate is further digested, the lower molecular weight chains will provide small peptides, i.e. very low molecular weight digests, and even tripeptides or dipeptides depending on the number of amino acid residues. Collagen hydrolysate is also known to be used as a dietary supplement for athletes with activity-related joint pain. See Clark et al., “24-Week study on the use of collagen hydrolysate as a dietary supplement in athletes with activity-related joint pain” a Cuff. Med. Res. Opin., 2008 May; 24(5):1485-96.

### SUMMARY OF THE INVENTION

The present disclosure is directed to an injectable composition comprising a Cathepsin K blocking amount of a polydisperse preparation of collagen macropeptides. In embodiments, the average molecular weight the polydisperse preparation of collagen macropeptides is between about 10 and about 100 kDa, formulated in an acceptable sterile injection vehicle. In embodiments, 90% by weight of the collagen macropeptides will have a molecular weight of less than 100 kDa. In further embodiments, 95% by weight of the collagen macropeptides will have a molecular weight of less than 100 kDa. In further embodiments, 90% of the collagen macropeptides have a molecular weight of less than 50 kDa. In further embodiments, 95% of the collagen macropeptides have a molecular weight of less than 50 kDa. In further embodiments, 90% of the collagen macropeptides have a molecular weight of less than 30 kDa. In other embodiments, 95% of the collagen macropeptides have a molecular weight of less than 50 kDa. In further embodiments, 95% of the collagen macropeptides have a molecular weight of less than 30 kDa.

The present disclosure is also directed to methods for treating arthritis, comprising administering an intra-articular injection of the composition of claim 1 into the joint of a patient in need of treatment thereof. In embodiments, the arthritis may be osteoarthritis, rheumatoid arthritis, psoriatic arthritis, or juvenile arthritis. “Patient” or “subject” as used herein refers to a mammalian subject diagnosed with or suspected of having or developing an arthritis, cartilage disorder, or other Cathepsin K related disease. In some embodiments, the term “patient” refers to a mammalian subject with a higher than average likelihood of developing arthritis, cartilage disorder, or other Cathepsin K related disease. Exemplary patients may be humans, apes, dogs, pigs, cattle, cats, horses, goats, sheep, rodents and other mammalians that may benefit from the therapies disclosed herein. Exemplary human patients may be male and/or female. “Patient in need thereof” or “subject in need thereof” is referred to herein as a patient diagnosed with or suspected of having a disease or disorder, for instance, but not limited to arthritis or osteoarthritis.

The present disclosure is further directed to a method of making the injectable compositions described herein comprising: (a) dissolving gelatin into an aqueous liquid to form a collagen macropeptide-containing liquid solution, (b) filtering the collagen macropeptide-containing liquid solution through a molecular weight cutoff filter configured to substantially remove collagen peptides larger than 100 kDa, (c) collecting the filtrate with macropeptides smaller than 100 kDa, and (d) formulating the filtrate with macropeptides smaller than 100 kDa with an acceptable sterile injection vehicle. The molecular weight cutoff filters used can comprise units which utilize centrifugal force, positive pressure filtration, tangential flow filtration, or other methods designed for such purpose. In further embodiments, the process further includes lyophilizing the collected filtrate for subsequent formulation prior to patient injection.

The present disclosure is further directed to an aqueous composition comprising a Cathepsin K blocking amount of collagen macropeptides with an average molecular weight between about 10 and about 100 kDa that is free flowing at room temperature and does not gel at 4° C.

The present disclosure further relates to an injectable composition comprising (1) a polydisperse preparation of collagen macropeptides with an average molecular weight of between about 10 and about 100 kDa, (2) a pharmaceutically active ingredient, (3) an acceptable sterile injection vehicle. The pharmaceutically active ingredient may be a small molecule, large molecule, or biologic molecule.

The present disclosure is further directed to a finished pharmaceutical dosage form comprising a polydisperse preparation of collagen macropeptides with an average molecular weight of between about 10 and about 100 kDa and a pharmaceutically active ingredient.

The present disclosure is yet further directed to an implantable medical device comprising a polydisperse preparation of collagen macropeptides with an average molecular weight of between about 10 and about 100 kDa and a pharmaceutically active ingredient.

### BRIEF DESCRIPTION OF THE DRAWINGS

FIG. 1 illustrates a centrifugal apparatus that may be used in preparing collagen macropeptides in accordance with the embodiments described herein.

FIG. 2A is a protein-stained SDS-PAGE gel showing molecular weight of collagen macropeptides yielded in the filtrate of dissolved gelatin using 100, 50, 30 and 10 kDa molecular weight cutoff filters.

FIG. 2B is a SDS-PAGE gel stained for total protein or detected by fluorescent imaging showing collagen macropeptides by molecular weight.

FIG. 3 depicts the results from an example assay for dose-dependent decrease of Cathepsin K collagenase activity by increasing dose of collagen macropeptides.

FIG. 4 are visible light and fluorescent imaging over 4 weeks demonstrating incorporation of fluorescent-labeled collagen macropeptides (Fluor-MCOL) into cartilage.

FIG. 5 illustrates a tangential flow apparatus that may be used in preparing collagen macropeptides in accordance with the embodiments described herein.

FIG. 6 illustrates a stirred cell apparatus that may be used in preparing collagen macropeptides in accordance with the embodiments described herein.

### DETAILED DESCRIPTION OF THE INVENTION

The present disclosure relates to a local (injectable) treatment to block Cathepsin K enzymatic activity and avoiding the adverse effects associated with systemic delivery of Cathepsin K inhibitors. We have discovered that collagen macropeptides of a particular molecular weight range are able to effectively block Cathepsin K enzymatic activity.

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting.

Although various features of the disclosure may be described in the context of a single embodiment, the features may also be provided separately or in any suitable combination. Conversely, although the present disclosure may be described herein in the context of separate embodiments for clarity, the present disclosure may also be implemented in a single embodiment. The following definitions supplement those in the art and are directed to the current application and are not to be imputed to any related or unrelated case, e.g., to any commonly owned patent or application. Although any methods and materials similar or equivalent to those described herein may be used in the practice for testing of the present disclosure, the preferred materials and methods are described herein. Accordingly, the terminology used herein is for the purpose of describing particular embodiments only, and is not intended to be limiting.

We have discovered that collagen macropeptides of a particular size block Cathepsin K enzymatic activity. It is believed that collagen macropeptides of the present disclosure act as a sacrificial substrate barrier for the collagenase enzyme Cathepsin K, and can therefore block degradation of intact helical collagen as would otherwise occur in diseases such as osteoarthritis. We have also developed a molecular weight filtration process in the preparation of collagen macropeptides in a simple process for sterilization, endotoxin removal, and elimination of solution thickening/gelling at temperatures as low as refrigeration. The collagen macropeptide filtrate can also be lyophilized and readily reconstituted by the addition of a desired injection vehicle (e.g., saline, water, etc) and at a desired concentration/strength.

Collagen macropeptides as described herein may be obtained as a filtrate of commercially available gelatins. Gelatin is extracted from animal tissues rich in collagen such as skin, sinews, and bone. Although it is possible to extract gelatin from these materials using boiling water, it is more practical to first pretreat the animal tissues with either acid or alkali. Gelatin obtained from the acid process is called type A, whereas gelatin obtained from the alkali process is called type B. Common sources of gelatin are bovine, porcine, or fish skins. There are numerous available sources of gelatin including, for example, from commercial suppliers such as Sigma-Aldrich, and from various gelatin manufacturers. A preferred type of gelatin is low bloom strength gelatin on the bloom strength scale of 30 to 325 with 30 being the softest (least viscous) and 325 being the stiffest (most viscous). Most preferably, low bloom strength gelatins are used with a bloom strength between 50-125.

The term “collagen macropeptides” as used herein, generally refers to peptides that are small enough to pass through a 100 kDa molecular weight cutoff filter. Preferred collagen macropeptides are derived from food or medical grade gelatin and will have an average molecular weight of about: 10 kDa to about 100 kDa, about 20 kDa to about 90 kDa, about 30 kDa to about 80 kDa, about 30 kDa to about 100 kDa, about 30 kDa to about 50 kDa, about 50 kDa to about 60 kDa, about 40 kDa to about 70 kDa, about 50 kDa to about 100 kDa, about 10-50 kDa, or about 10 kDa to about 30 kDa. Although some amount of the peptides comprising collagen macropeptides may be larger than about 100 kDa, it is preferable to have the majority of all collagen macropeptides in the composition fall below about 100 kDa, 50 kDa, 30 kDa, or 10 kDa.

In another aspect of the composition about 80% by weight of the collagen macropeptides have a molecular weight of less than about 100 kDa, about 85% by weight of the collagen macropeptides have a molecular weight of less than about 100 kDa, about 90% by weight of the collagen macropeptides have a molecular weight of less than about 100 kDa, about 95% by weight of the collagen macropeptides have a molecular weight of less than about 100 kDa, about 96% by weight of the collagen macropeptides have a molecular weight of less than about 100 kDa, about 97% by weight of the collagen macropeptides have a molecular weight of less than about 100 kDa, about 98% by weight of the collagen macropeptides have a molecular weight of less than about 100 kDa, or about 99% by weight of the collagen macropeptides have a molecular weight of less than about 100 kDa.

In another aspect of the composition, about 80% by weight of the collagen macropeptides have a molecular weight of less than about 50 kDa, about 85% by weight of the collagen macropeptides have a molecular weight of less than about 50 kDa, about 90% by weight of the collagen macropeptides have a molecular weight of less than about 50 kDa, about 95% by weight of the collagen macropeptides have a molecular weight of less than about 50 kDa, about 96% by weight of the collagen macropeptides have a molecular weight of less than about 50 kDa, about 97% by weight of the collagen macropeptides have a molecular weight of less than about 50 kDa, about 98% by weight of the collagen macropeptides have a molecular weight of less than about 50 kDa, or about 99% by weight of the collagen macropeptides have a molecular weight of less than about 50 kDa.

In another aspect of the composition, about 80% by weight of the collagen macropeptides have a molecular weight of less than about 30 kDa, about 85% by weight of the collagen macropeptides have a molecular weight of less than about 30 kDa, about 90% by weight of the collagen macropeptides have a molecular weight of less than about 30 kDa, about 95% by weight of the collagen macropeptides have a molecular weight of less than about 30 kDa, about 96% by weight of the collagen macropeptides have a molecular weight of less than about 30 kDa, about 97% by weight of the collagen macropeptides have a molecular weight of less than about 30 kDa, about 98% by weight of the collagen macropeptides have a molecular weight of less than about 30 kDa, or about 99% by weight of the collagen macropeptides have a molecular weight of less than about 30 kDa.

In accordance with the disclosure, average molecular weight and/or molecular weight distribution may be determined by standard techniques known to those of ordinary skill in the art including size exclusion chromatography with spectrophotometric detection and/or by gel electrophoresis (SDS-PAGE) with densitometric analysis of separated components.

A “Cathepsin K blocking amount” is meant to be an amount of collagen macropeptides which shows a significant blockade of the maximal enzymatic activity of Cathepsin K when measured using a Fluorescence Resonance Energy Transfer (FRET) peptide enzymatic assay or other assays known to those of ordinary skill in the art for quantifying peptide enzymatic activity. In the context of treating arthritis, a “Cathepsin K blocking amount” inhibits, reduces, or ameliorates arthritis or pain associated with arthritis or cartilage joint disorders, as determined by a clinician using standard means for diagnosis, prognosis or measuring pain on standardized pain scale. Such an assay comprises pre-incubation of various quantities of collagen macropeptides with a fixed quantity of purified Cathepsin K enzyme in an assay buffer containing sodium acetate, followed by incubation of the mixture with a collagen-mimetic FRET peptide and measurement of fluorescence intensity in a spectrofluorometer. In such an assay, maximal Cathepsin K activity is determined by performing a reaction in the absence of added collagen macropeptides. A Cathepsin K blocking amount of collagen macropeptides with an average molecular weight between 10 and 100 kDa may be an amount between about 0.5 to about 20.0 mg/ml, about 0.5 to about 15.0 mg/ml, about 0.5 to about 10.0 mg/ml, about 0.5 to about 5.0 mg/ml, about 5.0 to about 20.0 mg/ml, about 5.0 to about 15.0 mg/ml, about 5.0 to about 10.0 mg/ml, about 10.0 to about 20.0 mg/ml, or about 10.0 to about 15.0 mg/ml. Other collagen macropeptide concentrations could be utilized as well.

As the term “polydisperse preparation” of collagen macropeptides is used herein, it is intended to mean that there is a variety of peptide chain lengths (and attendant molecular weights) included within the collagen macropeptides. Thus, the composition described herein comprises a heterogeneous mixture of varying molecular weight collagen macropeptides.

The term “about” or “approximately” means within an acceptable error range for the particular value as determined by one of ordinary skill in the art, which will depend in part on how the value is measured or determined, i.e., the limitations of the measurement system. For example, “about” may mean within 1 or more than 1 standard deviation, per the practice in the art. Alternatively, “about” may mean a range of up to 20%, up to 10%, up to 5%, or up to 1% of a given value. In another example, the amount “about 10” includes 10 and any amounts from 9 to 11. In yet another example, the term “about” in relation to a reference numerical value may also include a range of values plus or minus 10%, 9%, 8%, 7%, 6%, 5%, 4%, 3%, 2%, or 1% from that value. Where particular values are described in the application and claims, unless otherwise stated the term “about” meaning within an acceptable error range for the particular value should be assumed.

An acceptable sterile injection vehicle can include one or more excipients suitable for use in injectable products. For example, the injection vehicle may include water, sodium chloride in water (saline), phosphate buffered saline (PBS), ethanol, propylene glycol, glycerin, antioxidants, chelating agents, emulsifier and suspending agents, lyophilization reagents, osmotic pressure regulators, viscosity modifiers pH modifiers, buffering agents, solubilizer, preservatives, antimicrobials, surfactants, polymers, and other adjuvants. Additional specific examples include polysorbate 80, propylene glycol, benzyl alcohol, citric acid, sodium citrate, and glycine. Lactose and mannitol are also frequently used in connection with a lyophilized product.

The collagen macropeptides of the present disclosure may be useful in treating any disease where preventing cartilage and/or collagen degradation provides a beneficial effect or blocking activity of the Cathepsin K enzyme or any collagenase activity would be beneficial. In particular, the collagen macropeptides of the present disclosure are useful in treating arthritis. More specifically, the compositions described herein are useful in treating osteoarthritis, rheumatoid arthritis, psoriatic arthritis (and/or plaque psoriasis) and juvenile arthritis. Other types of arthritis treatable with the compositions described herein include ankylosing spondylitis, reactive arthritis, infectious arthritis, enteropathic arthritis, degenerative joint disease, chondromalacia, stickler syndrome, osteochondrodysplasias, multiple epiphyseal dysplasia, achondroplasia, undifferentiated arthritis, other cartilage defects including ear piercings and reconstruction, and other connective tissue diseases or defects such as all forms of tendonitis, ruptures and tears. In one aspect, the administration may be an intra-articular administration to a joint or to an area of cartilage damage. In another aspect, the joint comprises a knee, wrist, shoulder, or hip joint. Any joint or area of the body impacted by arthritis or a cartilage disorder may be the area of administration.

Other conditions involving cathepsin K may also be treatable with compositions of the present invention. Such conditions include, for example, inflammation such as intra-plantar inflammation, tendinopathy, and wound healing.

Compositions of the present disclosure may further include compositions typically injected into joints to treat arthritis. For example, it may be advantageous to include chondroitin sulfate, glucosamine, hyaluronic acid, corticosteroids or other ingredients for the treatment of osteoarthritis.

The compositions of the present disclosure can be prepared by the following a process comprising:
a. dissolving gelatin into an aqueous liquid to form a collagen peptide containing liquid solution,
b. filtering the collagen containing liquid solution through a molecular cutoff filter configured to substantially remove collagen peptides larger than about 100 kDa,
c. collecting the filtrate with macropeptides smaller than about 100 kDa, and;
d. formulating the filtrate with macropeptides smaller than about 100 kDa with an acceptable sterile injection vehicle.

Step (a) includes dissolving the gelatin into an aqueous liquid to form a collagen peptide containing liquid solution. It may be desirable to heat the aqueous liquid, such as water or PBS, to facilitate dissolution. It may further be desirable to add other ingredients such as salts to facilitate dissolution of the gelatin. Upon dissolution of the gelatin in step (a), the solution will include collagen peptides of wide variety of amino chain lengths and molecular weights with an average molecular weight in excess of 100 kDa. Unfiltered gelatin solutions typically thicken at room temperature and may set firm when refrigerated.

In step (b), the collagen peptide containing liquid solution is filtered using a molecular weight cutoff filter typically varying in sizes of 10, 30, 50 and 100 kDa molecular weight cutoff sizes. Upon filtration, peptides in excess of the cutoff filter size are retained in the gelatin solution and the filtered collagen macropeptides smaller than the filter cutoff size are passed through into the filtrate. Unlike the unfiltered gelatin solution, the filtrate containing the collagen macropeptides will not substantially thicken or set at low temperatures and may be lyophilized and reconstituted in saline.

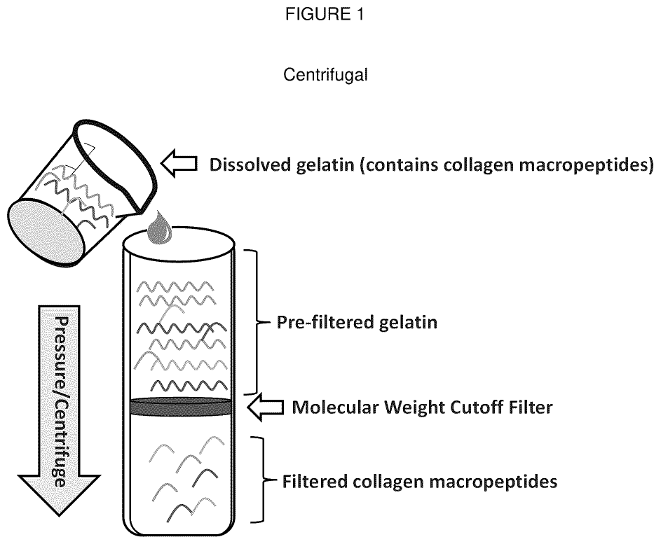

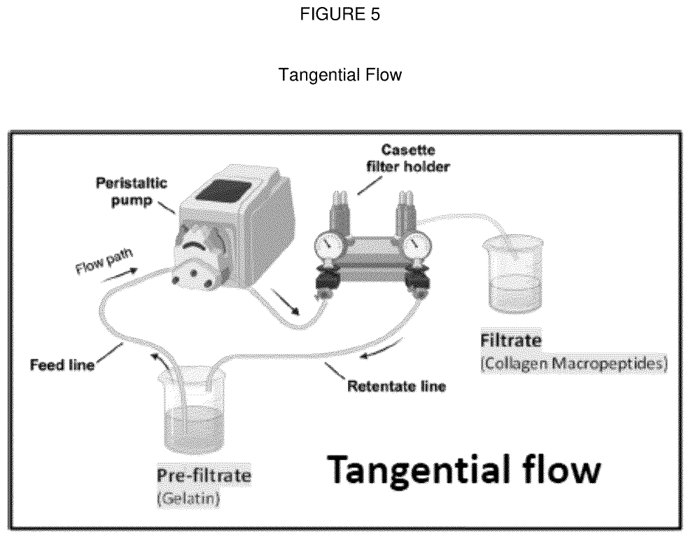

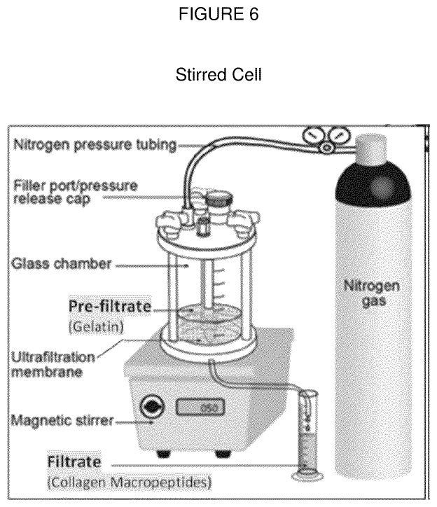

The filtrate is then collected and formulated with an acceptable sterile injection vehicle. The formulating step may be completed with the addition of water, saline, PBS or other excipients added at any prior point during the process such as dissolving step (a). The order of steps presented here and in the claims is not intended to be limiting. Other excipients as described above may constitute the injection vehicle or may be added to an injection vehicle such as water, PBS, or ethanol. Collagen macropeptide preparations may also be formulated such that they can be applied by a variety of routes, for example topically or parenterally. The process of the present disclosure may be conducted on any apparatus capable of separating out collagen macroparticles as described herein including, but not limited to, the apparatus depicted in each of FIGS. 1, 5 and 6.

Additional optional steps may include lyophilizing the before or after step (d) with reconstitution prior to patient administration, for example at the point of use, or in a physician's office.

The present disclosure further relates to an injectable composition comprising (1) a polydisperse preparation of collagen macropeptides with an average molecular weight of between about 10 and about 100 kDa, (2) a pharmaceutically active ingredient, (3) an acceptable sterile injection vehicle. The pharmaceutically active ingredient may be a small molecule, large molecule, or biologic molecule.

The pharmaceutically active ingredient may be selected from the group consisting of cortisone, betamethasone, betamethasone acetate, betamethasone dipropionate, betamethasone valerate, cortivasol, dexamethasone, dexamethasone acetate, dexamethasone sodium phosphate, hydrocortisone acetate, hydrocortisone sodium phosphate, hydrocortisone sodium succinate, hydrocortisone valerate, hydrocortisone aceponate, hydrocortisone butyrate, hydrocortisone cypionate, hydrocortisone proputate, methylprednisolone, methylprednisolone aceponate, methylprednisolone acetate, methylprednisolone sodium phosphate, methylprednisolone sodium succinate, prednisolone, prednisolone acetate, prednisolone, metasulfobenzoate, prednisolone sodium phosphate, prednisolone, steaglate, prednisolone tebutate, triamcinolone, triamcinolone acetonide, triamcinolone acetonide 21-palmitate, triamcinolone benetonide, triamcinolone diacetate, triamcinolone hexacetonide, alclometasone, alclometasone dipropionate, amcinonide, amelometasone, beclomethasone, beclomethasone dipropionate, monohydrate, budesonide, butixocort, butixocort propionate, ciclesonide, ciprocinonide, clobestasol, clobestasol propionate, clocortolone, clobestasone, clobestasone butyrate, clocortolone pivalate, cloprednol, cortisone, cortisone acetate, deflazacort, domorednate, deprodone, deprodone propionate, desonide, desoximethasone, desoxycotone, desoxycortone acetate, dichlorisone, diflorasone diflorasone diacetate, diflucortolone, difluprdnate, fluclorolone, fluclorolone acetonide, fludrocortisone flucortisone acetate fludroxycortide, flumethasone, flumethasone pivalate, flunisolide, fluocinolone, fluocinolone acetonide, flucortin, flucortolone, fluorometholone, fluticasone, fluticasone furoate, fluticasone propionate, fluorometholone acetate, fluoxymesterone, fluperolone, fluprednidene, fluprednidene acetate, fluprednisolone, formocortal, halcinonide, halobetasol propionate, halometasone, halopredone, halopredone acetate, hydrocortamate, isoflupredone, isoflupredone, acetate, itrocinonide, loteprednol etabonate, mazipredone, meclorisone, meclorisone dibutyrate, medrysone, meprednisone, mometasone, mometasone furoate, mometasone furoate monohydrate, nivacortol, paramethasone, paramethasone acetate, prednazoline, prednicarbate, prednisolone, prednylidene, procinonide, rofileponide, rimexoone, timobesone, tipredane, tixocortol, toxocortol pivalate, and tralonidel, or the like, and stereoisomers thereof, including racemates or enantiomers.

The pharmaceutically active ingredient may be one or more of aspirin, ibuprofen, naproxen, celecoxib, diclofenac, ketorolac, meloxicam, indomethacin, nabumetone, oxaprozin, etodolac, fenprofen, flurbiprofen, mefenamic acid, piroxicam, sulindac, tolmetin, methotrexate, sulfasalazine, leflunomide, hydroxychloroquine, prednisone, cortisone, hydrocortisone, methylprednisone, dexamethasone, betamethasone, deflazacort, triamcinolone, fludrocortisone, fluocinole, fluocinonide, and pharmaceutically acceptable salts thereof and stereoisomers thereof.

The pharmaceutically active ingredient may be one or more of baricitinib, tofacitinib, upadacitinib, etanercept, infliximab, adalimumab, golimumab, tocilizumab, secukinumab, ixekizumab, bimekizumab, brodalumab, sarilumab, ustekinumab, abatacept, celecoxib meloxicam apremilast, certolizumab pegol, deucravacitinib, filgotinib, olokizumab, ianalumab, semaglutide, bimekizumab, risankizumab, or biosimilars thereof.

The present disclosure is further directed to a finished pharmaceutical dosage form comprising a polydisperse preparation of collagen macropeptides with an average molecular weight of between about 10 and about 100 kDa and a pharmaceutically active ingredient. The finished pharmaceutical dosage form may further include with any standard physiologically and/or pharmaceutically acceptable carriers or excipients which are known in the art. The formulation compositions preferably are sterile and contain a therapeutically effective amount of the pharmaceutically active ingredient.

The present disclosure is yet further directed to an implantable medical device comprising a polydisperse preparation of collagen macropeptides with an average molecular weight of between about 10 and about 100 kDa and a pharmaceutically active ingredient. The medical device, such as an insert for a bony defect, may be formed from the collagen macropeptides with an active ingredient and any other implantable material.

### EXAMPLES

### Example I

Four compositions in accordance with the disclosure were prepared as follows: As depicted in FIG. 1, a commercially available gelatin was dissolved at 4% (40 mg/ml) in water. 0.4 ml samples were filtered through a 100 kDa cutoff membrane, a 50 kDa cutoff membrane, and 30 kDa cutoff membrane and a 10 kDa cutoff membrane. The filtrates were recovered and assayed for Cathepsin K blocking activity with a FRET peptide enzymatic assay. Protein concentrations were determined on an Invitrogen Nanodrop Spectrophotometer measuring absorbance at 205 nm. Samples were pre-incubated with Cathepsin K enzyme, transferred to a multi-well plate and then fluorescent-labeled collagen substrate (FRET substrate) was added to each well.

Fluorescence was then measured. The greater the Cathepsin K blocking activity of the collagen macropeptides, the greater the reduction in fluorescence. The data obtained corresponding to each filtrate is set forth in Table 1 below.

TABLE 1

| | Protein Concentration | % Blockade of CatK |
|---|---|---|
| Sample | (mg/ml) | Activity |
| Unfiltered | 35.1 | 31% |
| Gelatin* | | (1/10 dilution of |
| | | unfiltered solution) |
| 100 kDa filtrate | 5.88 | 70% |
| 50 kDa filtrate | 3.92 | 68% |
| 30 kDa filtrate | 3.31 | 57% |
| 10 kDa filtrate | 0.59 | 18% |

*Starting solution for filtration (gelatin from bovine skin, type B).

The dramatic increase in % blockade of Cat K activity at low protein concentration was unexpected.

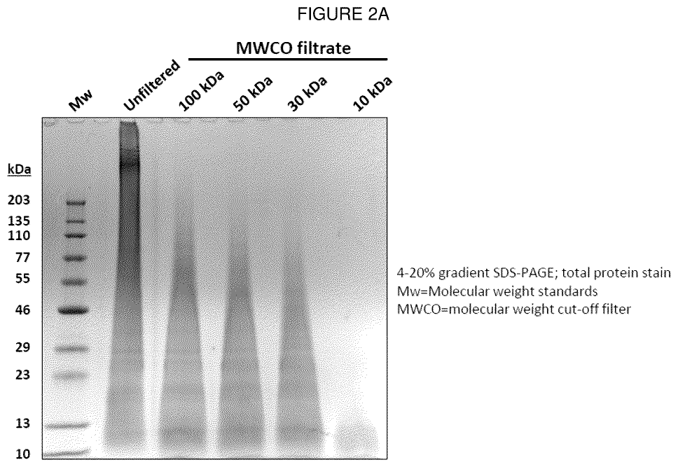

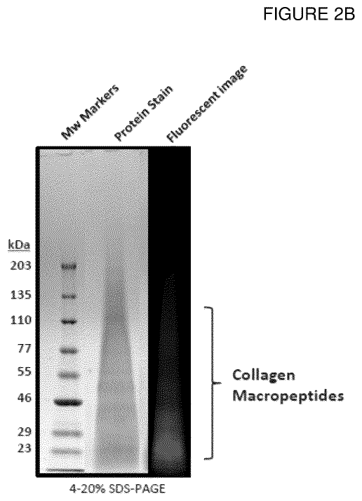

Samples stored at 4° C. were analyzed for molecular weight of collagen macropeptides yielded in the filtrate of dissolved gelatin using the 100, 50, 30 and 10 kDa molecular weight cutoff filters described above as reflected in FIG. 2. Gel electrophoresis (SDS-PAGE) with densitometric analysis of separated components is reflected in the Western Blot included in FIG. 2.

### Example II

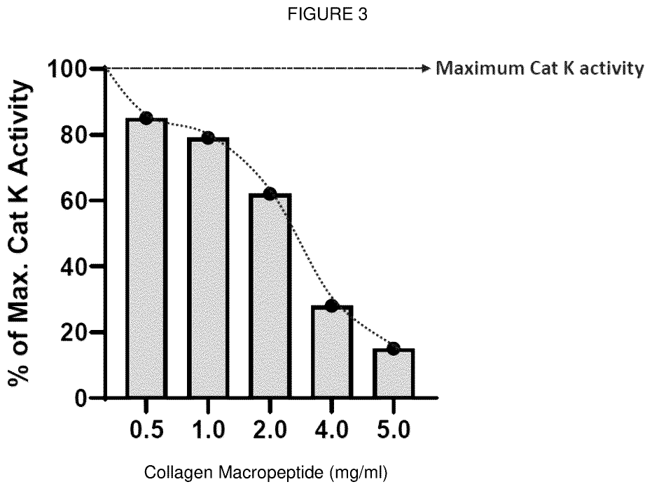

In a second experiment, dose-dependency decrease of Cathepsin K collagenase activity by increasing dose amounts of collagen macropeptides was tested. The samples were prepared by dissolving a commercially available gelatin at 4-5% (w/v; 40-50 mg/ml) in water and filtration through a 50 kDa cutoff membrane. Various concentrations of collagen macropeptides from dilutions of the filtrate were prepared for FRET assay. The % of maximum Cathepsin K activity vs. collagen macropeptide concentration for each sample is depicted in FIG. 3. The data shows Cathepsin K activity is reduced as the concentration of collagen macropeptides is increased.

### Example III

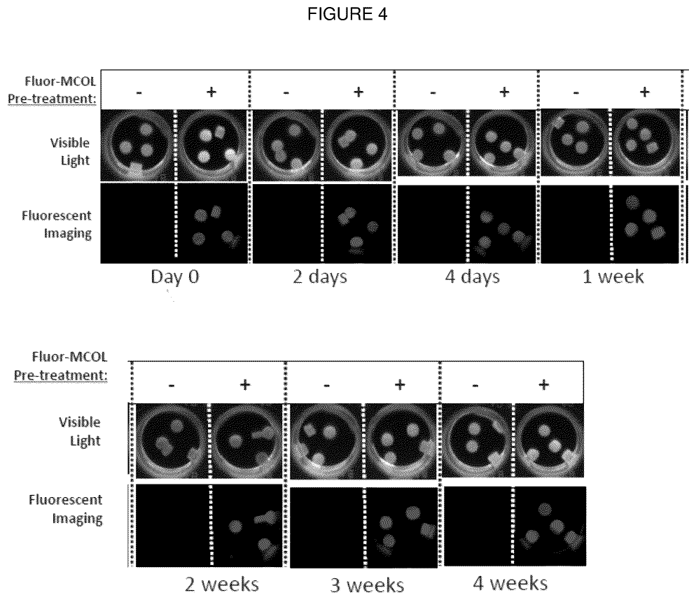

In a third example, it is demonstrated that collagen macropeptides (MCOL) as described herein are readily incorporated into cartilage and such collagen macropeptides remain incorporated in the cartilage over an extended period of time. FIG. 4 shows both visible light and fluorescent images of cartilage discs including control (−) discs that were exposed unlabeled MCOL in culture media and experimental cartilage discs (+) that were exposed to fluorescent labeled collagen macropeptides. (“Fluor-MCOL”). Both control (−) and experimental (+) discs were partially degraded to simulate OA. Control (−) discs were incubated with unlabeled MCOL in culture alone for 3 hours. Experimental (+) discs were incubated with culture media containing Fluor-MCOL (5 mg/ml). Cartilage was washed and then maintained in fresh culture media with periodic media changes. No additional Fluor-MCOL was added to the experimental (+) discs after incubation. M Fluorescent imaging was accomplished with an Invitrogen iBright FL1000 imager with fluorescence excitation at 665 nm and 688 nm. As seen in FIG. 4, Fluor-MCOL were readily incorporated into cartilage after 3 hours of incubation and remained substantially incorporated at 4 weeks.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

1. An injectable composition comprising a polydisperse preparation of collagen macropeptides with an average molecular weight of between about 10 and about 100 kDa and an acceptable sterile injection vehicle.

2. (canceled)

3. The composition of claim 1, wherein about 90% by weight of the collagen macropeptides have an average molecular weight of less than about 100 kDa.

4. (canceled)

5. The composition of claim 1, wherein about 90% of the collagen macropeptides have an average molecular weight of less than about 50 kDa.

6. (canceled)

7. The composition of claim 1, wherein about 90% of the collagen macropeptides have a molecular weight of less than about 30 kDa.

8. (canceled)

9. The composition of claim 1, wherein the injection vehicle comprises saline solution.

10. The composition of claim 1, wherein the sterile injection vehicle includes one or more of water, sodium chloride in water (saline), phosphate buffered saline (PBS), ethanol, propylene glycol, glycerin, antioxidants, chelating agents, emulsifier and suspending agents, lyophilization reagents, osmotic pressure regulators, viscosity modifiers pH modifiers, buffering agents, solubilizer, preservatives, antimicrobials, surfactants, polymers, and other adjuvants. Additional specific examples include polysorbate 80, propylene glycol, benzyl alcohol, citric acid, sodium citrate, or glycine.

11. The composition of claim 1, wherein the polydisperse preparation of collagen macropeptides is derived from gelatin.

12. The composition of claim 11 wherein the gelatin is animal derived.

13. The composition of claim 12 wherein the gelatin is bovine, porcine, or fish derived.

14. The composition of claim 11, wherein the gelatin is a low blooming gelatin.

15. The composition of claim 1, wherein a Cathepsin K blocking amount of collagen macropeptides with an average molecular weight between about 10 and about 100 kDa is between about 0.5 mg/ml to about 20.0 mg/ml.

16. A method for treating arthritis, comprising administering an intra-articular injection of the composition of claim 1 into the joint of a patient in need of treatment thereof.

17. The method of claim 16, wherein the joint is a knee, wrist, shoulder, or hip joint.

18. The method of claim 16, wherein the arthritis is osteoarthritis.

19. The method of claim 16, wherein the arthritis is rheumatoid arthritis.

20. The method of claim 16, wherein the arthritis is psoriatic arthritis.

21. The method of claim 16, wherein the arthritis is juvenile arthritis.

22. A method of making the composition of claim 1 comprising:
a. dissolving gelatin into an aqueous liquid to form a collagen peptide containing liquid solution,
b. filtering the collagen containing liquid solution through a molecular cutoff filter configured to substantially remove collagen peptides larger than about 100 kDa,
c. collecting the filtrate with macropeptides smaller than about 100 kDa, and;
d. formulating the filtrate with macropeptides smaller than about 100 kDa with an acceptable sterile injection vehicle.

23. The method of claim 22, further comprising lyophilizing the collected filtrate for subsequent formulation prior to patient injection.

24. The method of claim 22, further comprising heating the aqueous liquid thereby facilitating dissolution of the gelatin.

25.-33. (canceled)
